(19) United States
(12) Patent Application Publication
(10) Pub. No.: US 2025/0049991 A1
(43) Pub. Date: Feb. 13, 2025

(54) MICROELECTRODE CHIP FOR EFFICIENT DIFFERENTIATION OF NEURAL STEM CELLS AND METHOD FOR DIFFERENTIATING FUNCTIONAL NERVE CELLS USING SAME

(71) Applicant: Sogang University Research & Business Development Foundation, Seoul (KR)

(72) Inventors: Bong Geun CHUNG, Seoul (KR); Nayeon Kim, Seoul (KR); Yoon Young Choi, Seoul (KR); Tae Hyeon Kim, Seoul (KR); Jang Ho Ha, Seoul (KR)

(21) Appl. No.: 18/660,401

(22) Filed: May 10, 2024

(30) Foreign Application Priority Data
Aug. 9, 2023 (KR) 10-2023-0104090

(51) Int. Cl. A61L 27/36; A61L 27/38; C12M 1/42; C12M 3/06; C12N 5/0793; C12N 13/00

(52) U.S. Cl. CPC A61L 27/3691; A61L 27/383; C12M 23/16; C12M 35/02; C12N 5/0619; C12N 13/00; C12N 2501/13

(57) ABSTRACT

Disclosed herein are a microelectrode chip and a method for differentiating neural stem cells into neurons using same. The microelectrode chip can efficiently differentiate neural stem cells into neurons, thereby enabling the production of a large quantity of functional neurons through the differentiation of patient-specific neural stem cells. This facilitates quality-controlled production of neurons and can be widely used in various fields, including platforms for screening neural regeneration candidates and research into treatments for various degenerative neurological diseases.

### CROSS-REFERENCE TO RELATED APPLICATIONS

This application claims the benefit and priority to Korean Patent Application No. 10-2023-0104090, filed on Aug. 9, 2023. The entire disclosures of the applications identified in this paragraph are incorporated herein by references.

### SEQUENCE LISTING

This application contains references to amino acid sequences and/or nucleic acid sequences which have been submitted concurrently herewith as the sequence listing XML file entitled “000376 us_SequenceListing.XML”, file size 11,643 bytes, created on 9 May 2024. The aforementioned sequence listing is hereby incorporated by reference in its entirety pursuant to 37 C.F.R. § 1.52 (e) (5).

### FIELD

The present disclosure was carried out under the support of the Ministry of Science and ICT, with the unique project number 1711181531, sub-project number 2022R1A2C2003724. The research management agency for this project is the National Research Foundation of Korea, and the project is titled “Personal Basic Research (Ministry of Science and ICT)” with the research task named “Study on Differentiation of Nerve Cells from Reprogrammed Stem Cells Using Functional Electrical Stimulation Chips and Porous Nanomaterials.” The leading institution is Sogang University, and the research period spans from Mar. 1, 2024, to Feb. 29, 2025.

Additionally, the present disclosure was supported by the Ministry of Science and ICT under the unique project number 1711179437, sub-project number RS-2022-00070316. The research management agency for this project is the Inter-Ministerial Regenerative Medicine Development Project Team, and the project is named “Inter-Ministerial Regenerative Medicine Technology Development Project” with the research task titled “Development of Safety Assurance Technology Capable of Detecting and Removing Pre-differentiation Potency Stem Cells Adapted to Culture by Genomic Variation.” The leading institution is the Seoul National University Industry-Academic Cooperation Foundation, with the research period from Jan. 1, 2024, to Dec. 31, 2024.

Furthermore, the present disclosure was supported by the Ministry of Science and ICT under the unique project number 1711197702, sub-project number CP23008M. The research management agency for this project is the National Nanotechnology Center, and the project is named “Operation Cost Support (Major Business Cost) of the National Nanotechnology Center” with the research task titled “Development of Uncontact Digital PCR in Response to Next Wave (Post-Corona).” The leading institution is the National Nanotechnology Center, and the research period is from Jan. 1, 2024, to Dec. 31, 2024.

Furthermore, the present disclosure was supported by the Ministry of Science and ICT under the unique project number 1711198532, sub-project number RS-2023-00259341. The research management agency for this project is the National Research Foundation of Korea, and the project is named “Overseas excellent research institute cooperation hub establishment project” with the research task titled “Sogang-UPen Emerging Infectious Disease Theranostics Convergence Research Center.” The leading institution is Sogang University, and the research period is from Jan. 1, 2024, to Dec. 31, 2024.

The present disclosure relates to a microelectrode chip for the efficient differentiation of neural stem cells and a method for differentiating functional nerve cells using same. More specifically, the present disclosure is concerned with a method of differentiating functional nerve cells from human-derived neural stem cells using a microelectrode chip composed of cell culture channels and bridge channels interconnecting same.

### BACKGROUND

Neural stem cells (NSCs) are self-renewing cells that can proliferate indefinitely in an undifferentiated state and differentiate into neurons, astrocytes, and oligodendrocytes. The nervous system is essential for controlling most capabilities necessary for sustaining human life, necessitating extensive research into its mechanisms.

It is generally known that axons of neurons, which make up the peripheral nervous system, can regenerate and recover function after physical damage over a certain period. However, recovery to a normal state takes a long time, and in the case of the central nervous system, when neurons are damaged, they mostly fail to recover and permanently lose function.

Various methods have been researched for culturing and differentiating neural stem cells. However, there are unestablished technical limitations, such as the time required for differentiation, differences in efficiency of neural differentiation from neural stem cells according to protocols, and diversity in the rate of functional nerve cell differentiation from neural stem cells. Additionally, the complexity of the intracellular environment within actual neural networks exists, thus presenting challenges in replicating these complex neural networks.

To address these issues and limitations, research is actively ongoing to more precisely replicate the structure and function of in vivo nerves using microelectrode chips manufactured with microsystems, and to culture neural systems capable of simulating the central nervous system under various conditions. Microelectrode chips can be produced through simple processes, reduce the costs involved in experiments, and offer the advantage of yielding more accurate and promising data.

Utilizing such microelectrode chips to mimic human neural networks and to conduct research on axonal signal transmission, as well as studies on neural stem cells and other types of cells, is expected to be highly beneficial. These chips are anticipated to be widely used in various fields, including as platforms for screening potential neural regenerative substances and in research related to various degenerative neurological diseases. Furthermore, the use of microelectrode chips is expected to facilitate the production of quality-controlled, functional nerve cells in large quantities through the differentiation of patient-specific neural stem cells, thereby aiding in the production of neural cells.

### SUMMARY

The microelectrode chip, fabricated by the present inventors, including cell culture channels and bridge channels interconnecting same, was found to be efficient for differentiating neural stem cells into nerve cells, leading to the present disclosure.

The present disclosure aims primarily to provide a microelectrode chip for the differentiation of nerve cells, the microelectrode chip including:
a substrate containing a microelectrode and an electrode pad;
a first cell culture channel arranged on the substrate;
a second cell culture channel spaced apart in a parallel direction to the first cell culture channel; and
a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel.

Also, the present disclosure is to provide a method for differentiating neural stem cells into nerve cells using the microelectrode chip, the method including:
a microelectrode chip preparation step for preparing a microelectrode chip including a substrate containing a microelectrode and an electrode pad, a first cell culture channel arranged on the substrate, a second cell culture channel spaced apart in a parallel direction to the first cell culture channel, and a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel;
a loading step for loading neural stem cells into the first cell culture channel and the second cell culture channel; and
a differentiation step of differentiating the neural stem cells cultured in a medium containing a neurotrophic factor by applying electric stimulation to the microelectrode chip.

Furthermore, the present disclosure is to provide a method for screening a neural regeneration candidate substance using a microelectrode chip, the method including:
a microelectrode chip preparation step for preparing a microelectrode chip including a substrate containing a microelectrode and an electrode pad, a first cell culture channel arranged on the substrate, a second cell culture channel spaced apart in a parallel direction to the first cell culture channel, and a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel;
a loading step for loading neural stem cells into the first cell culture channel and the second cell culture channel;
a candidate substance contact step for contacting the neural stem cells with a candidate substance;
a differentiation step of differentiating the neural stem cells cultured in a medium containing a neurotrophic factor by applying electric stimulation to the microelectrode chip; and
a candidate substance verification step for assessing an extent of differentiation from neural stem cells into nerve cells and comparing the differentiation extent between cells contacted with and without the candidate substance.

The present disclosure relates to a microelectrode chip for the efficient differentiation of neural stem cells and a method for differentiation of nerve cells using same. The microelectrode chip according to the present disclosure enables efficient differentiation of neural stem cells into nerve cells.

A microelectrode chip composed of cell culture channels and bridge channels interconnecting same was fabricated by the present inventors, and it was found that the microelectrode chip can be effectively used in a method for differentiating nerve cells from neural stem cells.

Below, a detailed description will be given of the present disclosure.

An aspect of the present disclosure provides a microelectrode chip for the differentiation of nerve cells, the microelectrode chip including:
a substrate containing a microelectrode and an electrode pad;
a first cell culture channel arranged on the substrate;
a second cell culture channel spaced apart in a parallel direction to the first cell culture channel; and
a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel.

In the present disclosure, the nerve cells may include at least one selected from the group consisting of nerve fibers, dopamine neurons, and oligodendrocytes, but with no limitations thereto.

Another aspect of the present disclosure provides a method for differentiating neural stem cells into nerve cells using the microelectrode chip, the method including:

a microelectrode chip preparation step for preparing a microelectrode chip including a substrate containing a microelectrode and an electrode pad, a first cell culture channel arranged on the substrate, a second cell culture channel spaced apart in a parallel direction to the first cell culture channel, and a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel;
a loading step for loading neural stem cells into the first cell culture channel and the second cell culture channel; and
a differentiation step of differentiating the neural stem cells cultured in a medium containing a neurotrophic factor by applying electric stimulation to the microelectrode chip.

In the present disclosure, the neural stem cells may be induced neural stem cells (INSCs), but are not limited thereto.

In the present disclosure, the nerve cells may include at least one type of functional nerve cells selected from the group consisting of nerve fibers, dopamine neurons, and oligodendrocytes, but are not limited thereto.

In the present disclosure, the neurotrophic factor may be at least one type selected from the group consisting of brain-derived neurotrophic factor (BDNF), glial cell line-derived neurotrophic factor (GDNF), and sonic hedgehog, but with no limitations thereto.

In the present disclosure, the differentiation step may be carried out with electrical stimulation at a frequency of 1 to 4 Hz and a voltage of 300 to 700 mV for 3 to 7 minutes.

Preferably, the differentiation step may be carried out at a frequency of 1 to 3.5 Hz, 1 to 3 Hz, 1 to 2.5 Hz, 1.5 to 4 Hz, 1.5 to 3.5 Hz, or 1.5 to 3 Hz, for example, 1.5 to 2.5 Hz, but with no limitations thereto.

Preferably, the differentiation step may be carried out at a voltage of 300 to 650 mV, 300 to 600 mV, 350 to 700 mV, 350 to 650 mV, 350 to 600 mV, 350 to 550 mV, 400 to 700 mV, 400 to 650 mV, 400 to 600 mV, 400 to 550 mV, 450 to 700 mV, 450 to 650 mV, or 450 to 600 mV, for example, 450 to 550 mV, but with no limitations thereto.

The differentiation step is preferably carried out for 3 to 6 minutes, 4 to 7 minutes, for example, 4 to 6 minutes per day, but with no limitations thereto.

The differentiation step is preferably carried out over 7 days, but with no limitations thereto.

A further aspect of the present disclosure is to provide a method for screening a neural regeneration candidate substance using a microelectrode chip, the method including:
a microelectrode chip preparation step for preparing a microelectrode chip including a substrate containing a microelectrode and an electrode pad, a first cell culture channel arranged on the substrate, a second cell culture channel spaced apart in a parallel direction to the first cell culture channel, and a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel;
a loading step for loading neural stem cells into the first cell culture channel and the second cell culture channel;
a candidate substance contact step for contacting the neural stem cells with a candidate substance;
a differentiation step of differentiating the neural stem cells cultured in a medium containing a neurotrophic factor by applying electric stimulation to the microelectrode chip; and
a candidate substance verification step for assessing an extent of differentiation from neural stem cells into nerve cells and comparing the differentiation extent between cells contacted with and without the candidate substance.

The present disclosure relates to a microelectrode chip and a method for differentiating nerve cells from neural stem cells, which enables efficient differentiation of neural stem cells into nerve cells. With the ability to effectively differentiate nerve cells from neural stem cells, the microelectrode chip allows for the formation of form a large quantity of functional nerve cells through differentiation of patient-specific neural stem cells, aiding in the production of quality-controlled nerve cells. Furthermore, the microelectrode can find a wide spectrum of applications in various fields including platforms for screening neural regeneration candidate substances and research on the treatment of various degenerative neurological diseases.

### BRIEF DESCRIPTION OF THE DRAWINGS

The above and other aspects, features and advantages of the present disclosure will be more apparent from the following detailed description taken in conjunction with the accompanying drawings, in which:

FIG. 1A is a schematic diagram of a microelectrode chip according to an embodiment of the present disclosure.

FIG. 1B is a perspective view of a microelectrode chip according to an embodiment of the present disclosure.

FIG. 1C is an enlarged view of a microelectrode chip according to an embodiment of the present disclosure.

FIG. 2A shows a computational simulation result depicting the current density distribution of a microelectrode chip according to an embodiment of the present disclosure.

FIG. 2B is a graph showing the results of the current density distribution along the cross-sectional line a-a′ according to an embodiment of the present disclosure.

FIG. 2C is a graph depicting the relationship between the average current density in the bridge channel and the applied voltage according to an embodiment of the present disclosure.

FIG. 2D is a graph illustrating the current density in the bridge channel due to the direction of voltage according to an embodiment of the present disclosure.

FIG. 2E is a graph showing the shape of the average current density in the bridge channel caused by the applied voltage according to an embodiment of the present disclosure.

FIG. 3A is a diagram showing a protocol for culturing and differentiating nerve cells from human induced pluripotent stem cells (iPSCs) on a plate, according to an embodiment of the present disclosure.

FIG. 3B is a photograph showing a phase contrast image of induced neural stem cells (iNSCs) induced by neurotrophin factor (NF) on a plate, according to an embodiment of the present disclosure.

FIG. 3C is a photograph showing mRNA expression for neuronal-specific markers in iNSCs cultured on a plate, according to an embodiment of the present disclosure.

FIG. 3D is a graph showing mRNA expression for the neuronal-specific marker Oct4 in iNSCs cultured on a plate, according to an embodiment of the present disclosure.

FIG. 3E is a graph showing mRNA expression for the neuronal-specific marker Nestin in iNSCs cultured on a plate, according to an embodiment of the present disclosure.

FIG. 3F is a graph showing mRNA expression for the neuronal-specific marker PROX1 in iNSCs cultured on a plate, according to an embodiment of the present disclosure.

FIG. 3G is a graph showing mRNA expression for the neuronal-specific marker Olig2 in iNSCs cultured on a plate, according to an embodiment of the present disclosure.

FIG. 3H is a graph showing mRNA expression for the neuronal-specific marker TH in iNSCs cultured on a plate, according to an embodiment of the present disclosure.

FIG. 4A is a photograph showing immunofluorescence images of iNSCs cultured under conditions of electrical stimulation(ES), neurotrophin factor (NF), or their combined stimulation (ES+NF) for 7 days on a plate, according to an embodiment of the present disclosure.

FIG. 4B is a graph showing expression intensity of Tuj1 and PROX1 when differentiated under conditions of ES, NF, or ES+NF, according to an embodiment of the present disclosure.

FIG. 4C is a graph showing expression intensity of SV2 and PSD95 when differentiated under conditions of ES, NF, or ES+NF, according to an embodiment of the present disclosure.

FIG. 5A is a photograph showing immunofluorescence images of iNSCs cultured for 5 days on a microelectrode chip under conditions of ES, NF, or ES+NF, according to an embodiment of the present disclosure.

FIG. 5B is a graph showing expression intensity of Tuj1 and PROX1 when differentiated under conditions of ES, NF, or ES+NF, according to an embodiment of the present disclosure.

FIG. 6A is a photograph showing the impact of conditions ES, NF, or ES+NF on synapse formation through immunofluorescence images, according to an embodiment of the present disclosure.

FIG. 6B is a graph showing the number of synapses based on the density of SV2 and PSD95 under conditions of ES, NF, or ES+NF, according to an embodiment of the present disclosure.

FIG. 6C is a graph showing the relationship between the conditions of ES, NF, or ES+NF and the expression intensity of SV2 and PSD95, according to an embodiment of the present disclosure.

FIG. 7A is a photograph showing the impact of conditions ES, NF, or ES+NF on the differentiation of functional nerve cells through immunofluorescence images on a microelectrode chip, according to an embodiment of the present disclosure.

FIG. 7B is a graph showing the expression intensity of dopaminergic neurons (TH) and neurofilaments under conditions of ES, NF, or ES+NF, according to an embodiment of the present disclosure.

FIG. 7C is a graph showing the expression intensity of oligodendrocytes (Olig2) and Nestin under conditions of ES, NF, or ES+NF, according to an embodiment of the present disclosure.

### DETAILED DESCRIPTION

Hereinafter, embodiments of the disclosure will be described in detail in conjunction with the accompanying drawings. In the following description of the disclosure, a detailed description of known functions or configurations incorporated herein will be omitted when it may make the subject matter of the disclosure rather unclear.

It will be understood that when an element (e.g., first element) is “connected to” or “(operatively or communicatively) coupled with/to” to another element (e.g., second element), the element may be directly connected or coupled to another element, and there may be an intervening element (e.g., third element) between the element and another element.

On the other hand, it will be understood that when an element (e.g., first element) is “directly connected” or “directly coupled” to another element (e.g., second element), there is no intervening element (e.g., third element) between the element and another element. Other words used to describe the relationship between elements should be interpreted in a like fashion (e.g., “between” versus “directly between,” “adjacent” versus “directly adjacent,” etc.).

Throughout the description, the term “%” used to express the concentration of a specific material, unless otherwise particularly stated, refers to (wt/wt) % for solid/solid, (wt/vol) % for solid/liquid, and (vol/vol) % for liquid/liquid.

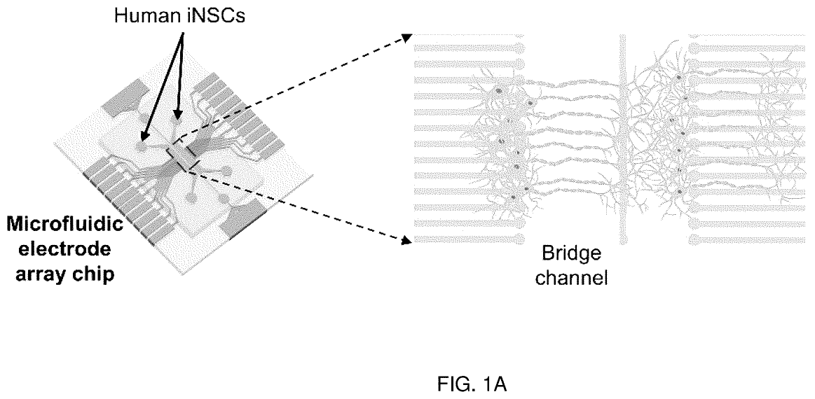

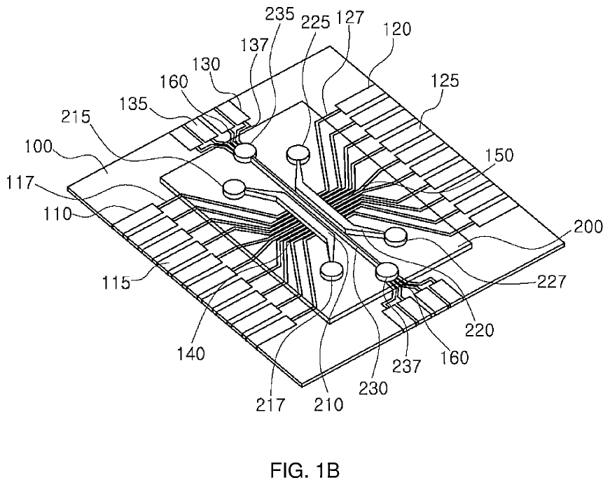

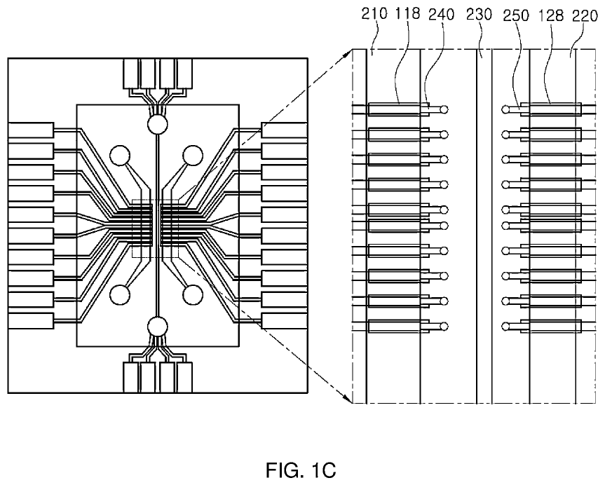

FIG. 1B is a perspective view of a microelectrode chip according to an embodiment of the present disclosure.

FIG. 1C is an enlarged view of a microelectrode chip according to an embodiment of the present disclosure.

Referring to FIGS. 1B or 1C, the microelectrode chip according to an embodiment may include a substrate 100 and microfluidic channels 200.

On the substrate 100, a first electrode pad section 110, a second electrode pad section 120, a third electrode pad section 130, a first electrode section 140, a second electrode section 150, and a third electrode section 160 may be arranged.

The first electrode pad section 110 may be located at one side on the substrate 100 and may include at least one first electrode pad 115. Each first electrode pad 115 may be connected to a first electrode line 117, allowing the first electrode pad 115 to receive current from an external source and supply same towards the first microelectrode 118 through the first electrode line 117.

The second electrode pad section 120 may be located at an opposite side on the substrate 100 and may include at least one second electrode pad 125. Each second electrode pad 125 may be connected to a second electrode line 127, allowing the second electrode pad 125 to receive current from an external source and supply same towards the second microelectrode 128 through the second electrode line 127.

The third electrode pad section 130 may be located at one side neighboring either the first electrode pad section 110 or the second electrode pad section 120 on the substrate 100, and may include at least one electrode pad 135. Each third electrode pad 135 may be connected to a third electrode line 137, allowing the third electrode pad 135 to receive current from an external source and supply same towards the third electrode line 137 through the third electrode line 137.

A plurality of microelectrodes 118 and 128 may be electrically connected to a single electrode line 117, 127. For example, 1 to 10, 1 to 9, 1 to 8, 1 to 7, 1 to 6, 2 to 10, 3 to 10, 4 to 10, 5 to 10, 6 to 10, 2 to 9, 3 to 7, 4 to 6, 1 to 30, 5 to 30, 10 to 30, 15 to 30, 20 to 30, 1 to 25, 1 to 20, 1 to 15, or 1 to 10 microelectrodes may be electrically connected to a single electrode line.

The first electrode section 140 may be positioned at the center of the substrate 100 and may include at least one first microelectrode 118 that are electrically connected to the first electrode pad section 110.

The current supplied from the first electrode pad 115 of the first electrode pad section 110 can flow through the first microelectrode 118, thereby enabling electrical stimulation to be applied to the nerve cells within the first cell culture channel 210 located above the first microelectrode 118.

The second electrode section 150 may also be positioned at the center of the substrate 100 and may include at least one second microelectrode 128 that is electrically connected to the second electrode pad section 120.

The second microelectrode 128 may be positioned to face the first microelectrode 118. In this regard, different poles may be connected to the first electrode pad 115 and the second electrode pad 125, respectively, thus allowing the current to flow in a specific direction between the first microelectrode 118 and the second microelectrode 128. Specifically, if the anode (+) is connected to the first electrode pad 115 and the cathode (−) to the second electrode pad 125, the current can consistently flow from the first microelectrode 118 towards the second microelectrode 128. Above the area between the first microelectrode 118 and the second microelectrode 128, the first bridge channel 240 and the second bridge channel 250 may be positioned. When the current flows in a consistent direction between the first microelectrode 118 and the second microelectrode 128, the bridge channels 240 and 250 will receive electrical stimulation from the unidirectional current. The influence of the unidirectional current in the bridge channels 240 and 250 facilitates the growth or differentiation of the nerve cells' axons along the bridge channels 240 and 250, thus promoting the growth and differentiation process of the nerve cells.

The second microelectrode 128 may receive current supplied from the second electrode pad 125 in the second electrode pad section 120, and thus, electrical stimulation may be applied to the nerve cells within the second cell culture channel 220 located above the second microelectrode 128.

The third electrode section 160 may be positioned between the third injection part 315 or the third discharge part 317 of the third cell culture channel 230 and the third electrode pad section 130, allowing for electrical connection therebetween.

The microfluidic channel 200 may include the first cell culture channel 210, the second cell culture channel 220, the third cell culture channel 230, one or more first bridge channels 240, and one or more second bridge channels 250.

The first cell culture channel 210, the second cell culture channel 220, and the third cell culture channel 230 may be supplied with a culture medium containing nerve cells and neurotrophic factors.

The first cell culture channel 210 may include a first injection part 215 to which a culture medium containing nerve cells and neurotrophic factors is supplied, and a first discharge part 217 from which the same culture medium is discharged.

The second cell culture channel 220 may include a second injection part 225 to which a culture medium containing nerve cells and neurotrophic factors is supplied, and a second discharge part 227 from which the same culture medium is discharged.

The third cell culture channel 310 may include a third injection part 315 to which the culture medium containing nerve cells and neurotrophic factors is supplied, and a third discharge part 317 from which the same culture medium is discharged.

The first cell culture channel 210 and the second cell culture channel 220 may be positioned parallel to each other and spaced apart, with the third cell culture channel 230 positioned therebetween.

A first bridge channel 240 may be located between the first cell culture channel 210 and the third cell culture channel 230, and a second bridge channel 250 may be located between the second cell culture channel 220 and the third cell culture channel 230.

The bridge channels 240 and 250 can function as pathways for the growth of axons as the nerve cells develop, allowing for the separation of axons from the nerve cells.

The bridge channels 240 and 250 may have a width and height that are challenging for the neuronal body to pass through but suitable for the penetration of axons. These channels may be designed to have a width and height corresponding to the diameter of the axons.

Therefore, as nerve cells in the first cell culture channel 210 receive neurotrophic factors and electrical stimulation and begin to grow, only the axons can pass through the bridge channels 240 and 250. As the axons grow along the bridge channels 240 and 250, the nerve cells may be oriented and fixed along the direction in which the axons are aligned.

The bridge channels 240 and 250 may have dimensions corresponding to the diameter of the axons of the nerve cells being cultured. For instance, the width and height of the bridge channels 240 and 250 may range from 1 to 20 μm, 1 to 18 μm, 1 to 16 μm, 1 to 14 μm, 1 to 12 μm, 1 to 10 μm, 3 to 20 μm, 5 to 20 μm, 7 to 20 μm, 9 to 20 μm, 3 to 18 μm, 5 to 16 μm, 7 to 14 μm, 9 to 12 μm, 9 to 11 μm, or precisely 10 μm.

The one or more bridge channels 240 and 250 positioned between the first cell culture channel 210 and the second cell culture channel 220 may be aligned on the same axis as the respective microelectrodes 118 and 128. This arrangement allows for each bridge channel 240, 250 to have one microelectrode, thereby effectively applying electrical stimulation to the growing nerve cells and their axons through the microelectrodes.

### EXAMPLE 1: Fabrication and Current Density Analysis Simulation of Microelectrode Chip

### 1-1. Fabrication of Microelectrode Chip

A microelectrode chip designed for the efficient cultivation and differentiation of neural stem cells was developed. The design of the microelectrode chip was carried out using AutoCAD software (Autodesk, CA, USA). The master mold for the microfluidic channels, based on polydimethylsiloxane (PDMS), was fabricated on a silicon wafer using a two-step photolithography process with SU-8 negative photoresist (Microchem Corp., MA, USA). The bridge channels and cell culture channels were patterned to heights of 5 μm and 150 μm, respectively, and the bridge channels between each cell culture channel were controlled to widths of 300 μm and 75 μm.

The microelectrode chip was produced using an electron beam deposition process. A transparent glass wafer (iNexus, Inc., Korea) was coated with photoresist, exposed to ultraviolet light for patterning, and then chromium and gold were deposited using an E-beam Evaporator to thicknesses of 5 nm and 50 nm, respectively. Unwanted portions of the photoresist were removed from the glass wafer using ethanol. A PDMS-based microfluidic channel mold was prepared using a silicone elastomer and curing agent (Sylgard 184, Dow Corning) at a 10:1 ratio. The microfluidic channels were sterilized with ethanol, rinsed three times with distilled water, and then the microfluidic channels and the electrode-patterned glass were bonded using oxygen plasma treatment (Femto Science, Korea). The assembly was then baked at 80° C. for one hour. The fabricated microelectrode chip was sterilized with 70% ethanol, washed three times with phosphate-buffered saline (PBS) (Gibco, MA, USA), air-dried overnight, and finally, cells were seeded thereto.

### 1-2. Computational Simulation for Current Density Analysis

The distribution of current density within the microfluidic channels was investigated using the AC/DC module of COMSOL Multiphysics 6.0 software (COMSOL, MA, USA). The simulation utilized the COMSOL Model Builder with a two-dimensional 2D) drawing sketched in AutoCAD, configuring the bridge channels and cell culture channels at heights of 5 μm and 150 μm, respectively. To determine the current density distribution, calculations were conducted in steady state using Ohm's law as follows:

J=σE=E/ρ

(J: current density, E: electric field, σ: electrical conductivity of the material, which is reciprocal number of the resistivity (ρ)).

In the simulation setup, a square wave modulation pulse was applied to one side of the gold pattern at a frequency of 0.5 Hz and an amplitude of ±0.5 V, while the other side of the gold pattern was grounded.

### EXAMPLE 2: Neural Stem Cell Culture and Differentiation

2-1. Culturing Human Induced Pluripotent Stem Cell (iPSC)

Thawed human iPSC WTC cells (passage 50-60 were cultured in mTeSR™1 culture medium (Stem Cell Technologies, CA, USA) on a 6-well plate coated with 1% Geltrex (Thermo Fisher Scientific, MA, USA). The medium was changed daily. After culturing the cells with ReLeSR® reagent (Stem Cell Technologies, CA, USA) for 4 minutes until they reached 80-90% confluency, the cells were washed twice with PBS and then passaged. The cells were pipetted into a new 6-well plate coated with Geltrex at a 1:3 split ratio.

Before plating the cells, the cell pellet was washed with mTeSR™1 medium (Stem Cell Technologies, CA, USA) supplemented with 10 μm Rho-associated kinase (ROCK) inhibitor Y-27632 (Tocris Bioscience, Bristol, UK) to enhance cell survival. After 24 hours, the medium was replaced with ROCK inhibitor-free mTeSR™1 medium.

2-2. Cultivation of Induced Neural Stem Cells (INSCs) from Human iPSCs

To generate three-dimensional (3D) spheroids, iPSCs were dissociated using ReLeSR™ reagent (Stem Cell Technologies, CA, USA) and plated on a non-adherent plate to enhance cell aggregation. At the time of plating (day 1), cells were seeded into clusters of approximately 80 to 100 cells per cluster in mTeSR™1 medium (Stem Cell Technologies, BC, CA). The mTeSeR1 medium was replaced daily until day 8, and on day 9, the floating spheroids were transferred to a tissue culture plate containing Dulbecco's Modified Eagle's Medium (DMEM)/F12 (Life Technologies, CA, USA) supplemented with ITS (Stem Cell Technologies, CA, USA) and 20 ng/ml fibronectin (Life Technologies, CA, USA) to promote adhesion and growth. On day 8 of proliferation, cell clones forming a well in a 6-well plate were mechanically scraped to create floating fragments. These fragments were plated on a plate coated with 50 μg/mL poly-L-ornithine (Sigma Aldrich, MO, USA) and 0.5 mg/mL laminin (Sigma Aldrich, MO, USA), along with a neural induction medium supplemented with 20 ng/ml brain-derived neurotrophic factor (BDNF) (R&D Systems, MN, USA), 200 ng/ml glial cell line-derived neurotrophic factor (GDNF) (R&D Systems, MN, USA), and 100 ng/ml Sonic hedgehog (Shh) (R&D Systems, MN, USA) as neurotrophic factors (NF). For the subsequent 12 days of neural differentiation, the cells were cultured with these neurotrophic factors, and the medium was replenished daily.

### 2-3. Immunofluorescence and Image Analysis

iNSCs (10, 30, and 50 days) and neurons cultured on the microelectrode chip (neural induction for 7 days) were washed with PBS and then fixed in 4% paraformaldehyde (Sigma Aldrich, MO, USA) for 30 minutes. The fixed cells were washed twice with PBS and permeabilized with 0.1% Triton X-100 (Samchun, Korea) dissolved in PBS for 30 minutes at room temperature. Non-specific binding proteins were blocked with 3% bovine serum albumin (BSA) (Sigma Aldrich, MO, USA) in PBS for one hour at room temperature. After washing with PBS, each sample was incubated with respective antibodies. The antibodies used were Anti-Tuj1 (1:1000, Biolegend, CA, USA), Anti-PROX1 (1:200, Abcam, Cambridge, UK), Anti-SV2 (1:200, Abcam, Cambridge, UK), Anti-PSD95 (1:200, Abcam, Cambridge, UK), Anti-Neurofilament (1:200, Sigma Aldrich, MO, USA), Anti-TH (1:200, Sigma Aldrich, MO, USA), Anti-NESTIN (1:200, Abcam, Cambridge, UK), and Anti-Olig2 (1:200, Abcam, Cambridge, UK) diluted in PBS, and the cells were incubated overnight at 4° C. The samples were gently rinsed with PBS and incubated overnight at 4° C. with secondary antibodies Alexa Fluor 488 goat anti-mouse IgG (1:200, Invitrogen, MA, USA) and Alexa Fluor 594 donkey anti-rabbit IgG (1:200, Invitrogen, MA, USA). All samples were counterstained with 4′,6-diamidino-2-phenylindole (DAPI, 1 mg/mL diluted in staining solution) for 10 minutes at room temperature. Immunostained images were acquired using a confocal laser scanning microscope (LSM710, Carl Zeiss, Jena, Germany). The fluorescence intensity of confocal images was analyzed using Image J software.

### 2-4. Reverse Transcription Polymerase Chain Reaction (RT-PCR)

Following the manufacturer's instructions, total RNA was extracted from differentiated neurons using TRIzol (Invitrogen, MA, USA). The RNA concentration was measured using NanoDrop (MicroDigital Co., Ltd., Korea). cDNA was synthesized from 1 μg of total RNA using the PrimeScript™ 1^{st} strand cDNA Synthesis Kit (TAKARA, Japan) according to the manufacturer's instructions. PCR products were separated by electrophoresis on a 2% (w/v) agarose gel in 1× TBE (Tris-borate-EDTA) buffer at 130V for 0.5 hours and stained with 0.5 μg/ml ethidium bromide. 1 microliter of the cDNA reaction mixture was amplified using gene-specific primers and AccuPower® PCR PreMix (BIONEER, Korea). A 100 bp ladder (TAKARA, Japan) was also loaded on each gel, and images were captured using an Axygen® Gel Documentation System (Coming, NY, USA). Band quantification was performed using Image J software. The mean and standard deviation of all experiments were calculated after normalizing the expression of each target gene to β-actin.

TABLE 1
Primers Used for Expression Analysis by PCR

| SEQ ID | Target | | SEQ ID | Target | |
|---|---|---|---|---|---|
| NO: | mRNA | Forward primer | NO: | mRNA | Reverse primer |
| 1 | Oct4 | GGAGGAAGCTGACAAC | 2 | Oct4 | GGCCTGCACGAGG |
| | | AATGAAA | | | GTTT |
| 3 | Nestin | TGGCCACGTACAGGAC | 4 | Nestin | CTTGGGGTCCTGAA |
| | | CCTC | | | AGCTGAG |
| 5 | PROX1 | GACTTTGAGGTTCCAG | 6 | PROX1 | TGTAGGCAGTTCGG |
| | | AGAGA | | | GGATTTG |
| 7 | TH | ACTGGTTCACGGTGGA | 8 | TH | TCTCAGGCTCCTCA |
| | | GTTC | | | GACAGG |
| 9 | Olig2 | CAGAAGCGCTGATGGT | 10 | Olig2 | TCGGCAGTTTTGGG |
| | | CATA | | | TTATTC |
| 11 | β-actin | AGCACAGAGCCTCGCC | 12 | β-actin | CATCATCCATGGTG |
| | | TT | | | AGCTGG |

### 2-5. Statistical Analysis

Each experiment was conducted at least in triplicate to ensure the reproducibility and reliability of the results. All results and data are presented as mean±standard deviation or standard error of the mean, and statistical analyses were performed using Student's t-test or one-way analysis of variance (ANOVA) with Bonferroni's multiple comparison test (GraphPad Prism version 8.0, GraphPad Software Inc., CA, USA). Differences between groups were considered statistically significant at *p<0.05, **p<0.01, and *** p<0.001. The black line above the histogram represents the comparison between the two groups.

### EXAMPLE 3: Neural Stem Cell Differentiation

### 3-1. Electrical Stimulation

To investigate neural differentiation, electrical stimulation(ES) was applied to the microelectrode chip. The chip was sterilized by UV irradiation and then incubated with 70% ethanol at room temperature for 30 minutes. Electrical stimulation was administered by applying a potential sequentially to each electrode for 5 minutes per day at a frequency of 2 Hz for 7 days. The electrical stimulation used was a biphasic square pulse ranging from 500 mV to −500 mV.

### 3-2. Differentiation of Induced Neural Stem Cells (INSCs)

After coating the microelectrode chip with 50 μg/mL poly-L-ornithine and 0.5 mg/mL laminin, iNSCs were loaded into the first and second cell culture channels. Subsequently, the iNSCs were cultured in DMEM/F12 neural induction medium containing 1% N2 and 2% B27 supplements, enriched with 20 ng/ml BDNF, 200 ng/mL GDNF, and 100 ng/ml Sonic hedgehog neurotrophic factors. The culture medium was replaced on the first day after loading and subsequently changed daily.

### Test Example 1: Verification of Current Density Analysis in Microelectrode Chip Structure

The microelectrode chip was composed of three cell culture channels: the first, second, and third cell culture channels, which were interconnected by bridge channels. The bridge channels created differences in fluid resistance, preventing the leakage of nerve cells into adjacent channels.

Current density plays a crucial role in inducing cell migration and promoting neural connections, which are important for the differentiation of neural stem cells. To understand and control current density, a computational simulation for analysis of current density was performed.

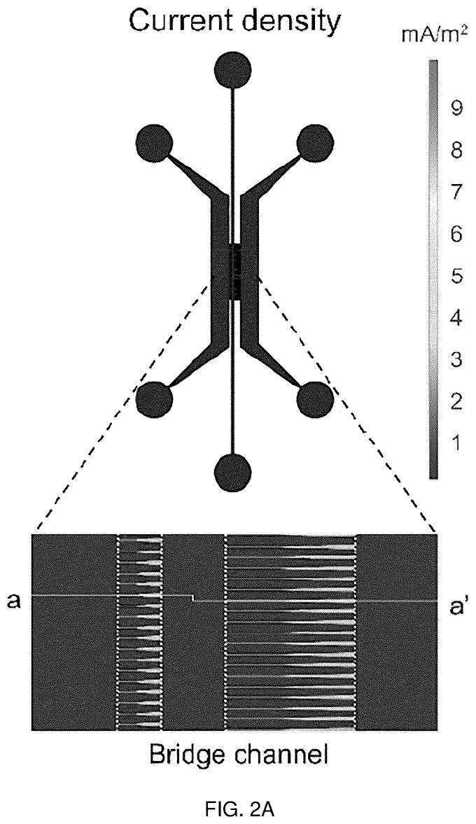

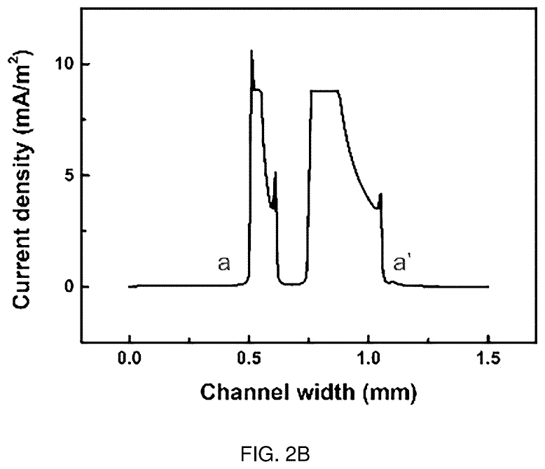

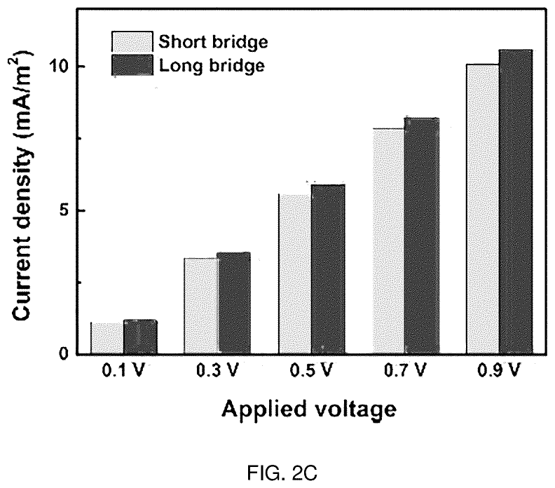

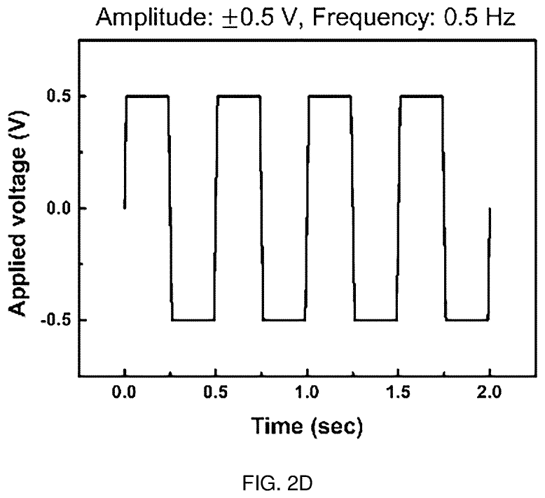

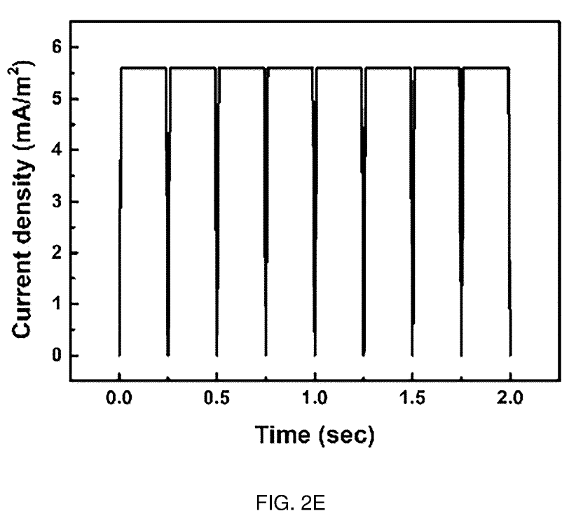

As can be seen in FIGS. 2A and 2B, the analysis of current density distribution along cross-section a-a′ showed that the current density within the bridge channels was higher compared to the cell culture channels. This difference was attributed to the narrower cross-sectional area of the bridge channels, and the higher current density in the bridge channels was observed to potentially guide the directionality of neural stem cell differentiation.

As seen in FIG. 20, the length of the bridge channels did not affect the average current density, and showed a linear proportionality to the applied voltage.

As shown in FIG. 2D, the current density within the bridge channels remained consistent regardless of the direction of the voltage.

As indicated in FIG. 2E, the current density in the bridge channels formed continuous pulses with intermittent breaks.

These findings suggest the potential for developing neurobiological applications based on controlled modulation of current density in microelectrode chips designed for neural stem cell differentiation.

### Test Example 2: Verification of Neural Stem Cell Cultivation

Before conducting experiments on the microelectrode chip, a four-step protocol was used for the cultivation and differentiation of neurons from human iPSCs

iPSC clones were cultured for 7 days to form embryoid bodies (EBs), and neural tube-like rosette structures appeared on the 8th day, with an increasing number of rosettes observed as the culture period extended. Furthermore, when iPSC-derived neural stem cells were plated on wells pre-coated with poly-L-ornithine and laminin, and cultured in serum-free medium supplemented with neurotrophic factors (NF), they formed neuronal-like cells and network-like structures that could persist for over a month.

To investigate whether iPSCs cultured in serum-free medium supplemented with NF could efficiently differentiate into neural stem cells, groups treated with neurotrophic factors and a spontaneous differentiation group (control) were examined. Cells were collected at 10, 30, and 50 days.

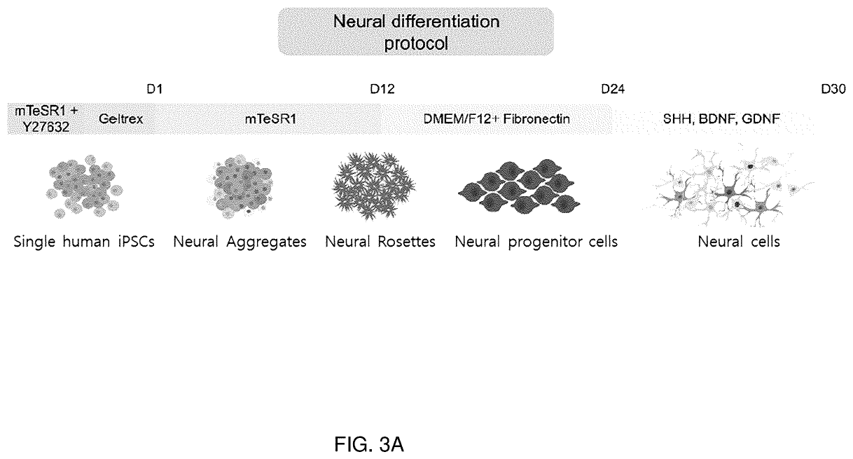

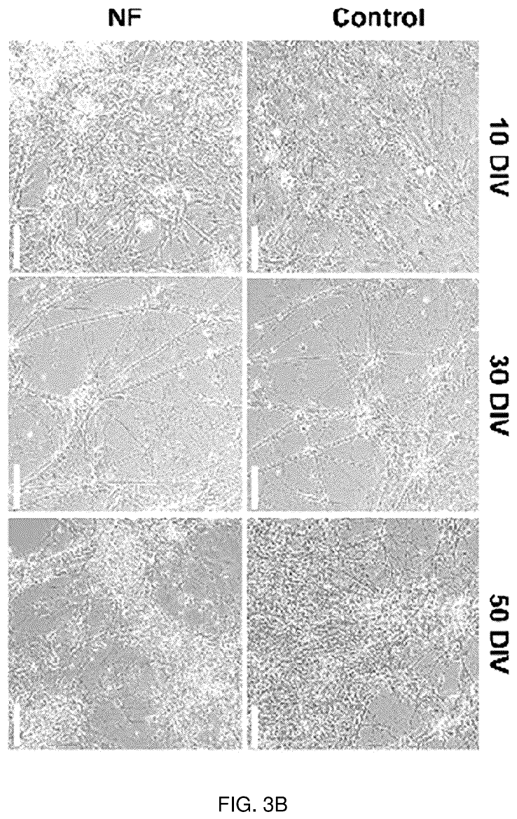

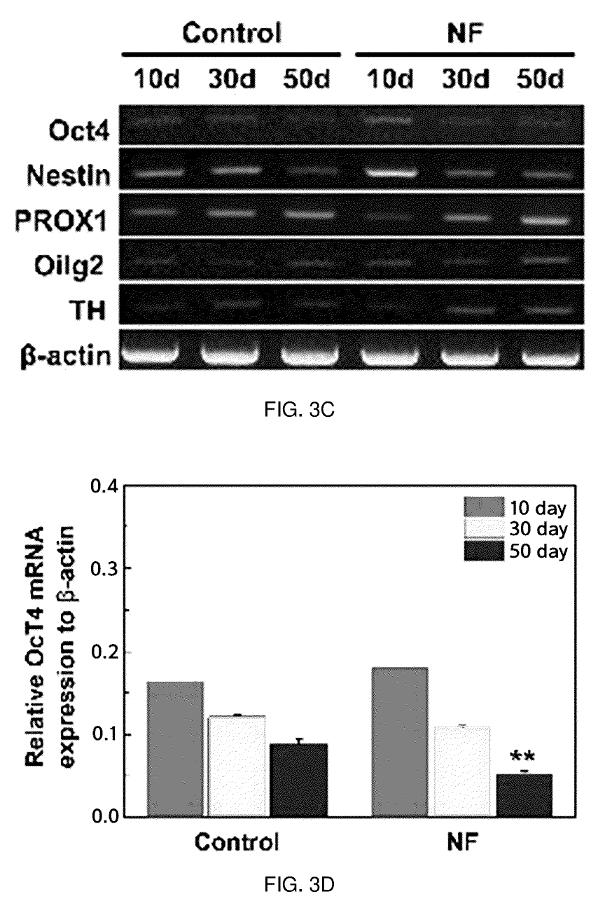

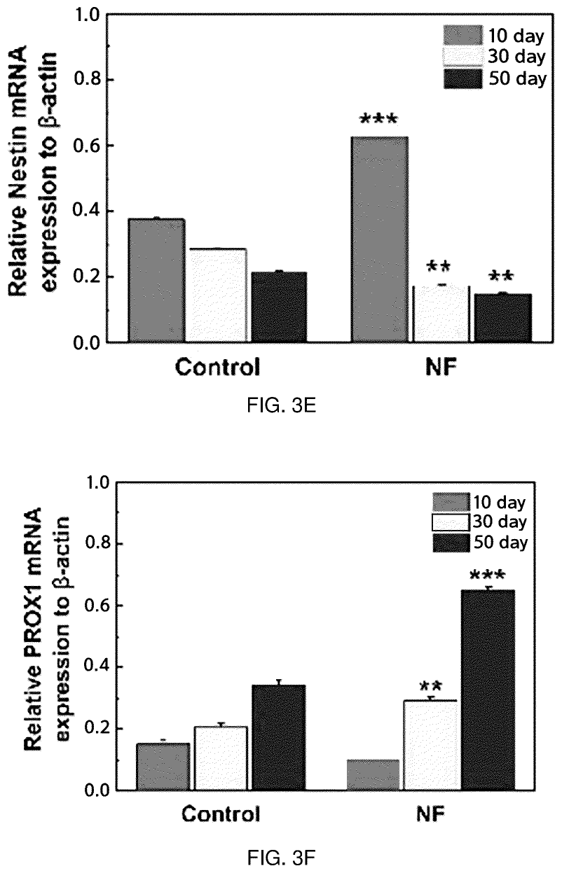

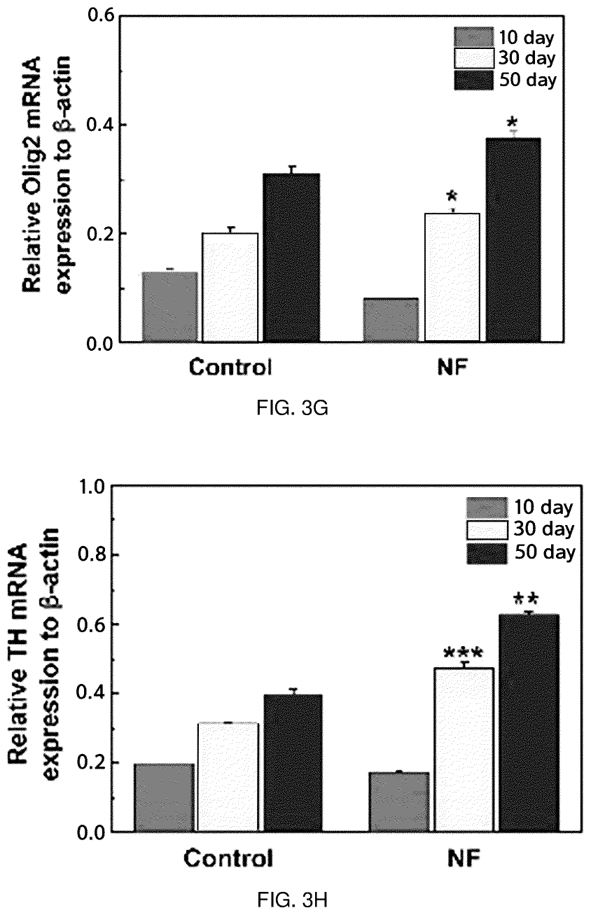

As shown in FIGS. 3C to 3H, RT-PCR results indicated that the expression levels of the pluripotency-related gene Oct4 were significantly lower in the neurotrophic factor-treated group compared to the control group, and a downward regulation was observed over time in all groups. In the control group, there was no significant change in the expression of Nestin, a marker protein used for neural stem cells, at 10 and 30 days, but a significant decrease was observed at 50 days. In the neurotrophic factor-treated group, the expression of nestin significantly decreased at 30 and 50 days compared to 10 days. Moreover, the expression of functional neuronal markers such as PROX1, Olig2, or TH was significantly higher in the neurotrophic factor-treated group than in the control group up to 50 days. Notably, PROX1, a marker specific to hippocampal neurons, showed the highest expression among all functional neuronal markers.

These results indicate that as cells transition from iPSCs to iNSCs, neural differentiation is proceeding normally in both the neurotrophic factor-treated group and the control group, with the potential for further differentiation into functional neurons.

### Test Example 3: Verification of Neural Stem Cell Differentiation by Electrical Stimulation(ES) and Neurotrophic Factors (NF)

### 3-1. Verification of Neuronal Differentiation on Plates

Initially, to investigate the effects of electrical stimulation(ES), single stimulation with neurotrophic factors (NF), or combined stimulation thereof (ES+NF) on iNSC differentiation, differentiation of NSCs under various stimulation conditions was performed on conventional culture plates. Neural differentiation was assessed by evaluating cell morphology or by analyzing the expression of neuronal-specific markers such as Tuj1, PROX1, and synaptic-specific markers like SV2 (presynaptic) and PSD95 (postsynaptic). Immunofluorescence images of cells differentiated on culture plates demonstrated that the cells had differentiated and matured under conditions of ES, NF, or ES+NF.

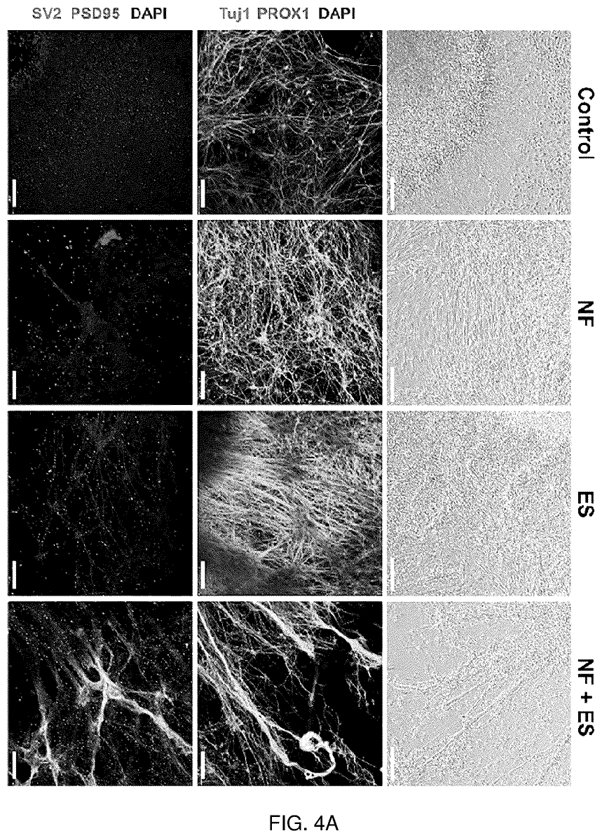

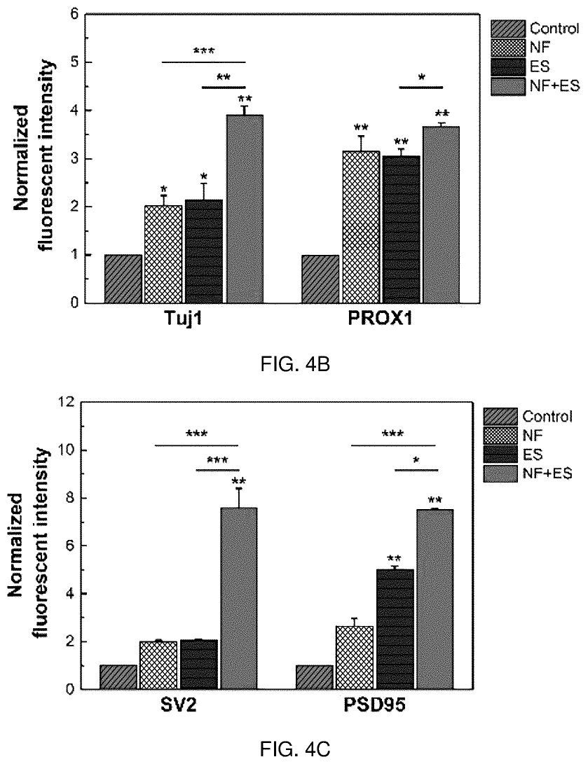

As observed in FIGS. 4A to 4C, immunofluorescence staining with Tuj1 and PROX1 on the 7th day of culture on the plates confirmed neuronal differentiation, with the most significant improvement seen in the ES+NF group. Additionally, an analysis of the fluorescence intensity of SV2 and PSD95 across all groups revealed that the values for SV2 and PSD95 were statistically significantly higher in the ES+NF treatment group.

Numerous studies have demonstrated that during neural differentiation, electrical stimulation(ES) accelerates the expression of neurotrophic factors (NF). Based on these findings, we have confirmed that ES not only increases endogenous NF but also effectively enhances neural differentiation and maturation in groups treated with both ES and exogenous NF.

As observed in FIG. 4A, immunofluorescence staining analysis showed no alignment of neurons in the control and NF groups; however, alignment was observed in the groups treated with ES and both NF and ES. Typically, during the differentiation process of neural stem cells, cells grown on unstructured substrates tend to grow in random directions. However, these results demonstrate that the current applied through ES induces alignment of neurons.

This leads to the discovery that the combination of ES and NF can enhance the differentiation and maturation of iNSCs into neurons in a more controlled manner.

### 3-2: Verification of Neuronal Differentiation on a Microelectrode Chip

Following the demonstration in Test Example 3-1 that the combination of neurotrophic factors (NF) and electrical stimulation(ES) has a synergistic effect on the differentiation and maturation of neural stem cells in plate cultures, we explored whether the effects of the stimulation conditions applied on plates could be monitored and quantified using a microelectrode chip. The impact of NF+ES stimulation on neuronal differentiation and maturation was analyzed through immunofluorescence staining, and the intensity of neural markers was quantified.

iNSCs were loaded onto the microelectrode chip and stimulated with ES, NF, or a combination of ES+NF to enhance maturity for up to 5 days. After differentiation, the identity of iPSC-derived neurons was confirmed on the fifth day by analyzing the expression of specific neuronal markers.

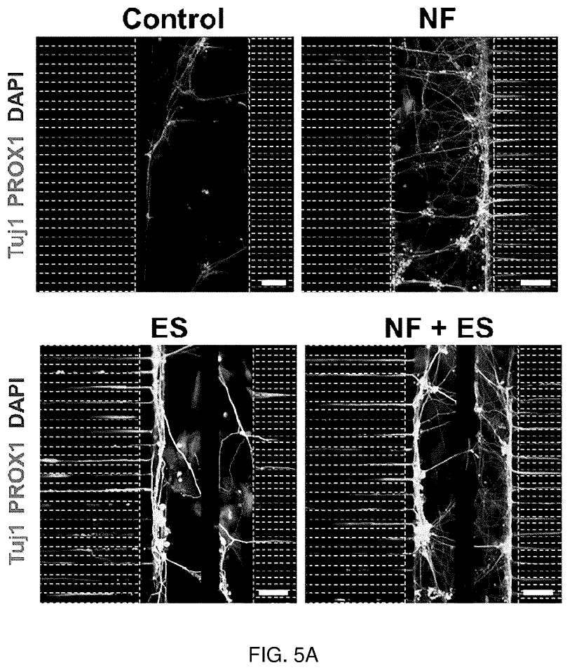

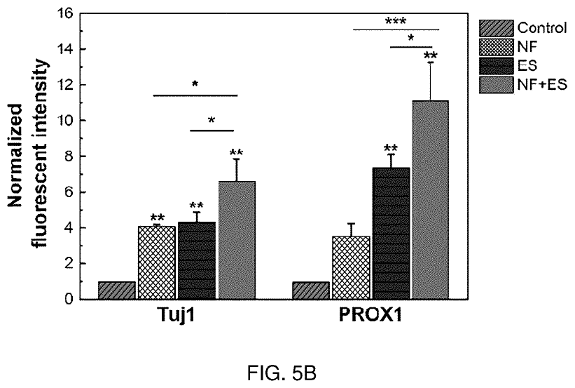

As observed in FIGS. 5A and 5B, the expression of Tuj1 and PROX1 was higher in the groups treated with ES, NF, or ES+NF compared to the control group.

Furthermore, PROX1, known to be expressed in hippocampal neurons as well as in muscle satellite cells, and the co-staining of Tuj1/PROX1 indicated differentiation of iPSCs into hippocampal neurons.

During neuronal differentiation, synapse formation plays a crucial role in establishing functional neural circuits, facilitating information processing, promoting functional specialization, regulating neurotransmitter signaling, and enhancing synaptic plasticity and circuit refinement. Therefore, it was analyzed whether the combination of ES+NF could regulate synapse formation and control the differentiation of neural stem cells into neurons. Synapse formation was verified by quantifying the fluorescence intensity of presynaptic marker SV2 and postsynaptic marker PSD95.

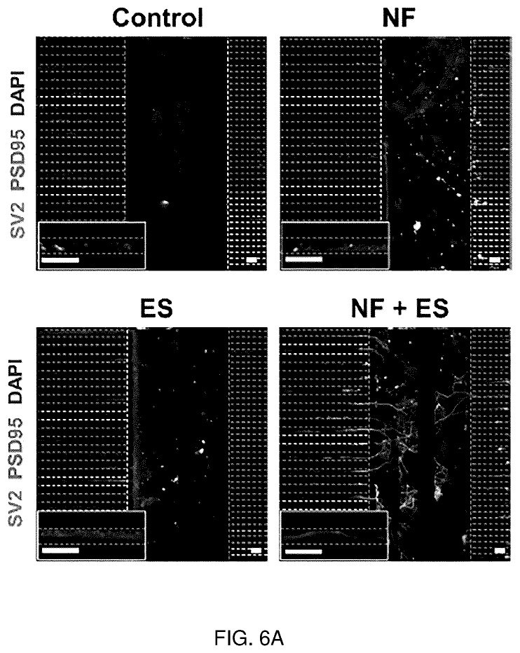

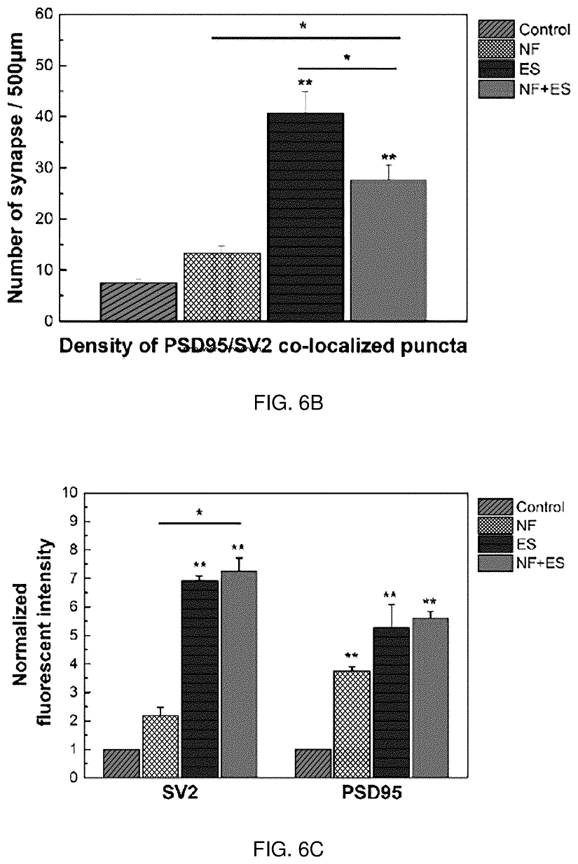

As seen in FIG. 6A, characteristic immunofluorescent spots of SV2 were more distinctly detected in neurons stimulated with ES, NF, or ES+NF on the fifth day compared to the control group.

FIGS. 6B and 6C demonstrate that the increase in SV2 expression indicates the formation of more synapses between adjacent neurons and the establishment of neural networks, signaling neuronal maturation. Treatment with ES+NF significantly increased the density of synaptic terminals (approximately 7.2 times that of the control group) and postsynaptic protrusions (approximately 5.6 times that of the control group) as indicated by SV2 and PSD95, respectively.

These findings confirm that the combination of ES+NF enhances both presynaptic and postsynaptic components, promotes synapse formation, and potentially improves the proliferation, differentiation, and maturation of iNSCs.

### 3-2-1. Verification of Functional Neuronal Differentiation on Microelectrode Chip

In the present disclosure, it was evaluated whether the use of a microelectrode chip with combined electrical stimulation(ES) and neurotrophic factors (NF) assists in the differentiation of human iNSCs into functional neurons. Specifically, the differentiation into distinct functional cell types such as neurofilament-bearing cells, dopaminergic neurons (TH), and oligodendrocytes (Olig2) was analyzed.

After stimulating iNSCs with ES, NF, or a combination of ES+NF for five days on the microelectrode chip, efficient differentiation into functional neurons was observed.

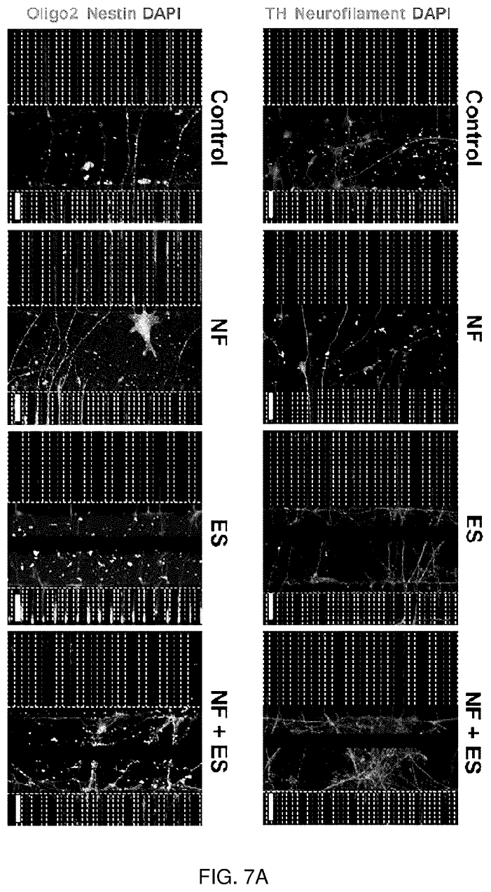

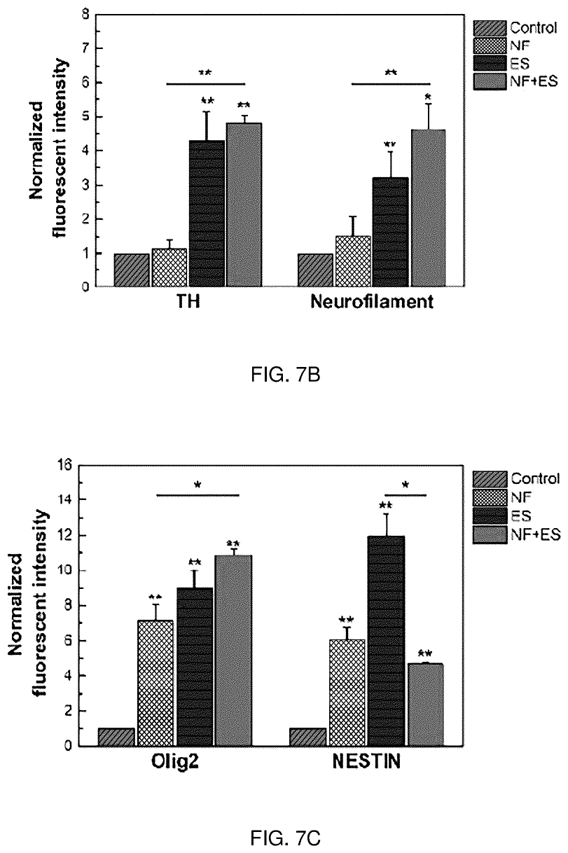

As shown in FIGS. 7A to 7C, immunocytochemical analysis revealed that the number of cells showing positive staining for neurofilament in the ES+NF treatment group was approximately five times higher than in the control group, the number of cells positive for TH was about five times higher, and the number of cells positive for Olig2 was about eleven times higher, indicating a significantly greater degree of differentiation compared to groups treated with either ES or NF alone.

However, the expression of nestin, a protein used as a marker for neural stem cells, significantly decreased in the ES+NF treatment group compared to other groups. Nestin is known to be expressed in the early stages of neural development, including in oligodendrocyte precursor cells (OPCs), and its expression is progressively downregulated as OPCs mature into oligodendrocytes. Therefore, the high expression of nestin observed in the groups treated with either ES or NF alone suggests the presence of a population of OPCs that have not yet fully differentiated into mature oligodendrocytes.

These observations confirm that the combination of ES+NF has a potent effect in promoting the differentiation of iPSCs into functional neurons.

What is claimed is:

1. A microelectrode chip for differentiation of neural stem cells, comprising:
a substrate containing a microelectrode and an electrode pad;
a first cell culture channel arranged on the substrate;
a second cell culture channel spaced apart in a parallel direction to the first cell culture channel; and
a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel.

2. The microelectrode chip of claim 1, wherein the nerve cells comprise at least one type of cells selected from the group consisting of nerve fibers, dopamine neurons, and oligodendrocytes.

3. A method for differentiating neural stem cells into nerve cells using the microelectrode chip, the method comprising:
a microelectrode chip preparation step for preparing a microelectrode chip including a substrate containing a microelectrode and an electrode pad, a first cell culture channel arranged on the substrate, a second cell culture channel spaced apart in a parallel direction to the first cell culture channel, and a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel;
a loading step for loading neural stem cells into the first cell culture channel and the second cell culture channel; and
a differentiation step of differentiating the neural stem cells cultured in a medium containing a neurotrophic factor by applying electric stimulation to the microelectrode chip.

4. The method of claim 3, wherein the neural stem cells are induced neural stem cells (INSCs).

5. The method of claim 3, wherein the nerve cells comprise at least one type of functional nerve cells selected from the group consisting of nerve fibers, dopamine neurons, and oligodendrocytes.

6. The method of claim 3, wherein the neurotrophic factor is at least one type selected from the group consisting of brain-derived neurotrophic factor (BDNF), glial cell line-derived neurotrophic factor (GDNF), and sonic hedgehog.

7. The method of claim 3, wherein the differentiation step is carried out with electrical stimulation at a frequency of 1 to 4 Hz and a voltage of 300 to 700 mV for 3 to 7 minutes.

8. A method for screening a neural regeneration candidate substance using a microelectrode chip, the method comprising:
a microelectrode chip preparation step for preparing a microelectrode chip including a substrate containing a microelectrode and an electrode pad, a first cell culture channel arranged on the substrate, a second cell culture channel spaced apart in a parallel direction to the first cell culture channel, and a third cell culture channel positioned between the first cell culture channel and the second cell culture channel,
wherein the first cell culture channel includes at least one first bridge channel that extends toward the third cell culture channel from a side adjacent to the third cell culture channel, and the second cell culture channel includes at least one bridge channel that extend toward the third cell culture channel from a side adjacent to the third cell channel;
a loading step for loading neural stem cells into the first cell culture channel and the second cell culture channel;
a candidate substance contact step for contacting the neural stem cells with a candidate substance;
a differentiation step of differentiating the neural stem cells cultured in a medium containing a neurotrophic factor by applying electric stimulation to the microelectrode chip; and
a candidate substance verification step for assessing an extent of differentiation from neural stem cells into nerve cells and comparing the differentiation extent between cells contacted with and without the candidate substance.


SEQUENCE LISTING (12 sequences)

SEQ ID NO: 1 - DNA, 23 bp, synthetic construct
  source 1..23: mol_type = other DNA
ggaggaagctgacaacaatgaaa

SEQ ID NO: 2 - DNA, 17 bp, synthetic construct
  source 1..17: mol_type = other DNA
ggcctgcacgagggttt

SEQ ID NO: 3 - DNA, 20 bp, synthetic construct
  source 1..20: mol_type = other DNA
tggccacgtacaggaccctc

SEQ ID NO: 4 - DNA, 21 bp, synthetic construct
  source 1..21: mol_type = other DNA
cttggggtcctgaaagctgag

SEQ ID NO: 5 - DNA, 21 bp, synthetic construct
  source 1..21: mol_type = other DNA
gactttgaggttccagagaga

SEQ ID NO: 6 - DNA, 21 bp, synthetic construct
  source 1..21: mol_type = other DNA
tgtaggcagttcggggatttg

SEQ ID NO: 7 - DNA, 20 bp, synthetic construct
  source 1..20: mol_type = other DNA
actggttcacggtggagttc

SEQ ID NO: 8 - DNA, 20 bp, synthetic construct
  source 1..20: mol_type = other DNA
tctcaggctcctcagacagg

SEQ ID NO: 9 - DNA, 20 bp, synthetic construct
  source 1..20: mol_type = other DNA
cagaagcgctgatggtcata

SEQ ID NO: 10 - DNA, 20 bp, synthetic construct
  source 1..20: mol_type = other DNA
tcggcagttttgggttattc

SEQ ID NO: 11 - DNA, 18 bp, synthetic construct
  source 1..18: mol_type = other DNA
agcacagagcctcgcctt

SEQ ID NO: 12 - DNA, 20 bp, synthetic construct
  source 1..20: mol_type = other DNA
catcatccatggtgagctgg